(19) United States
(12) Patent Application Publication
(10) Pub. No.: US 2009/0253996 A1
(43) Pub. Date: Oct. 8, 2009

(54) Integrated Sensor Headset

(76) Inventors: Michael J. Lee, Carmel, CA (US); Hans C. Lee, Carmel, CA (US)

Correspondence Address: COURTNEY STANIFORD & GREGORY LLP, P.O. BOX 9686, SAN JOSE, CA, 95157, US

(21) Appl. No.: 12/206,676

(22) Filed: Sep. 8, 2008

Related U.S. Application Data
(63) Continuation-in-part of application No. 11/681,265, filed on Mar. 2, 2007.
Application No. 11/681,265 is a continuation-in-part of application No. 11/804,517, filed on May 17, 2007.
(60) Provisional application No. 60/970,898, filed on Sep. 7, 2007.
(60) Provisional application No. 60/970,900, filed on Sep. 7, 2007.
(60) Provisional application No. 60/970,905, filed on Sep. 7, 2007.
(60) Provisional application No. 60/970,908, filed on Sep. 7, 2007.
(60) Provisional application No. 60/970,913, filed on Sep. 7, 2007.

(51) Int. Cl. A61B 5/0478

(52) U.S. Cl. 600/544

(57) ABSTRACT

A device is described that integrates sensors into a housing which can be placed on a human head for measurement of physiological data. The device includes at least one sensor and a reference electrode connected to the housing. A processor coupled to the sensor and the reference electrode receives signals that represent electrical activity in tissue of a user. The processor generates an output signal including data of a difference between an energy level in each of a first and second frequency band of the signals. The difference between energy levels is proportional to release level present time emotional state of the user. The device includes a wireless transmitter that transmits the output signal to a remote device. The device therefore processes the physiological data to create the output signal that correspond to a person's mental and emotional state (response).

### RELATED APPLICATIONS

This application is a continuation in part application of U.S. patent application Ser. No. 11/681,265, filed Mar. 2, 2007.

This application is a continuation in part application of U.S. patent application Ser. No. 11/804,517, filed May 17, 2007.

This application claims the benefit of U.S. Patent Application No. 60/970,898, filed Sep. 7, 2007.

This application claims the benefit of U.S. Patent Application No. 60/970,900, filed Sep. 7, 2007.

This application claims the benefit of U.S. Patent Application No. 60/970,905, filed Sep. 7, 2007.

This application claims the benefit of U.S. Patent Application No. 60/970,908, filed Sep. 7, 2007.

This application claims the benefit of U.S. Patent Application No. 60/970,913, filed Sep. 7, 2007.

### TECHNICAL FIELD

The disclosure herein relates generally to sensors. In particular, this disclosure relates to a sensor headset for gathering physiological data of a user wearing the headset.

### BACKGROUND

Devices used for sensing electrical activity in tissue have many uses in modern society. In particular modern electroencephalograms (EEGS) are used for measuring electrical activity in the brains of people for anesthesia monitoring, attention deficit disorder treatment, epilepsy prediction, and sleep monitoring, among other uses. Unfortunately, the complexity and cost of prior modern EEGs typically limits their use to clinics or other facilities where the device can be used on numerous people under the expert attention of a trained medical professional. Using the EEG on numerous people in a clinical setting helps to distribute the cost of the machine to the people which use it. EEGs can cost several thousand dollars.

Trained personnel are used for setting up and operating EEGs because of the complexities involved. Setting up prior EEGs involves preparing the skin of the person for connection of electrodes. The skin is typically prepared by shaving the hair from the area, sanding the skin to remove the outer surface and applying a conductive gel or liquid to the skin before attaching the electrode to the skin. Such extensive skin preparation is needed because contact resistance between the electrode and the skin must be reduced in order for prior EEGs to work properly. Contact resistance in these prior EEGs typically needs to be 20 k ohms or less.

Typical prior EEGs are subject to errors caused by electrical and magnetic noise from the environment surrounding the person. Errors are also caused by slight variations in internal components of the EEG and other sources, such as movement of the person during the operation of the EEG. Environmental noise can be caused by 60 Hz power in electrical wiring and lights in the area where the EEG is used, and other sources. Even the friction of any object moving through the air can cause noise from static electricity. Most or all prior EEGs have two electrodes are connected to the person's head and wires which are run from each of the electrodes to the EEG machine. The routing of the wires and the positions of the noise causing elements in the environment can cause significant errors in the measurements done by the EEG.

Measuring the electrical activity in the brain is difficult because the electrical signal being measured is many times smaller than the noise in the system. In many instances, the noise is on the order of a few volts or a few tens of volts while the electrical signal being measured is only in the microvolt range. This gives a signal-to-noise ratio of 10̂-6.

Prior EEGs have used very precise differential amplifiers, such as instrumentation amplifiers, to measure the electrical signal. The amplifier is referenced to a common reference such as the leg of the user. Each of the two wires from the two electrodes on the person's head are connected to the inputs of the differential amplifier. The output of the differential amplifier is a voltage relative to the reference which is proportional to the difference in voltage between the two electrodes times a constant. The measurement in this case is very sensitive because the differential amplifier is finding a small difference, the brain signal, between two signals which are 10̂6 times as large. These are reasons why small variations in components, the routing of the wires and other factors cause significant errors in the measurement and why prior EEGs are expensive and hard to use.

Another problem with the prior EEGs is that the 60 Hz noise is amplified at the first stage which saturates the signals before they are subtracted. In prior EEGs, designers go to great lengths to design systems that balance or shield the noise to avoid saturation. Systems which use the principle of subtracting two large numbers in measuring a small number are prone to these kinds of problems.

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### BRIEF DESCRIPTION OF THE DRAWINGS

FIG. 1 is an illustration of a system which uses a sensor device which measures electrical activity to determine a present time emotional state of a user.

FIG. 2 is an illustration of a program which contains a display of a level of the present time emotional state of the user and has controls for media material used in guiding the user in relation to the present time emotional state of the user.

FIG. 3 is a diagram of one example in which the media material guides the user based on the present time emotional state of the user.

FIG. 4 is a diagram of another example in which the media material guides the user based on the present time emotional state of the user.

FIG. 5 is a diagram of yet another example in which the media material guides the user based on the present time emotional state of the user.

FIG. 6 is a perspective view of the sensor device shown in FIG. 1.

FIG. 7 is a block diagram of the sensor device and a computer shown in FIG. 1.

FIG. 8 is a circuit diagram of an amplifier used in the sensor device shown in FIG. 7.

FIG. 9 is a circuit diagram of a filter stage used in the sensor device shown in FIG. 7.

FIG. 10 is a circuit diagram of a resistor-capacitor RC filter used in the sensor device shown in FIG. 7.

FIG. 11 is a circuit diagram of the amplifier, three filter stages and the RC filter shown in FIGS. 8, 9 and 10.

FIG. 12 is a block diagram of a digital processor of the sensor device shown in FIG. 7.

FIGS. 13a-13c show several views of a sensor headset, under an embodiment.

FIG. 14 is a flow chart for measuring physiological data using a sensor headset, under an embodiment.

### DETAILED DESCRIPTION

Physiological signals (data) of a person include but are not limited to heart rate, brain waves, electroencephalogram (EEG) signals, blink rate, breathing, motion, muscle movement, galvanic skin response, skin temperature, and any other physiological response of the person. Medical devices gathering physiological signals have existed for many decades and have become progressively more accurate through the development of new technologies. For a non-limiting example, head sensors use numerous electrodes placed around the head of an individual (a test subject or tester) to gather electrical signals from the person's brain. The resulting data can be collected and interpreted for many different uses including monitoring brain activity level and detecting sleep disorders. For another non-limiting example, non-invasive heart rate sensors are frequently used as diagnosis or monitoring tools in hospitals to track patients. These heart rate sensors are extremely useful as heart rate is one of the most critical components for the health of a patient. Many other medical grade physiological sensors are also being used in the medical field.

Many conventional physiological sensors currently in use are almost exclusively aimed at medical research and diagnosis and are complicated, bulky, expensive, and not user-friendly. These sensors are designed for the highest possible accuracy and data integrity. As a result, these sensors need to be operated by trained professionals in specific environments that limit any type of normal human activity. Consequently, the high cost and complexity of these sensors make them impractical to use outside of the medical field. For a non-limiting example, an EEG cap needs to cover the entire head of a person and has a long trail of wires connected to the data collection machine. This type of EEG requires preparing each skin contact point by abrading the skin and applying a conductive gel in order to guarantee a good high quality contact (and good conductivity). Such preparation process is both time consuming and painful due to the need to abrade the skin. Consequently, such device is very restrictive as it cannot be moved around easily with a person and does not allow the person to behave normally as he/she has a large cap attached to his/her head. For another non-limiting example, a non-invasive heart rate sensor is normally attached to a hand or foot of a person and is connected to a recording machine with a wire. Having this type of mechanism attached to a person's hand or foot would also preclude the person from behaving normally as he/she would be quite aware of the device hanging from his/her hand or fingers.

Physiological signals from a human body contain a wealth of information that has many uses beyond medical applications, and the integrated sensor headset described herein supports these uses. Advertisers, media producers, educators and other relevant parties have long desired to have greater feedback from their targets, customers, clients and pupils than simple surveys. A survey is potentially flawed due to the fact that a person has to think before responding to any inquiry, and various ideas, thoughts or experiences can affect their opinions and their responses to the survey. Using physiological sensors to gather data allows going under the layer or filter that is built into a person to obtain better understanding of the true reaction from the person to whatever stimuli being presented to him/her. Making this type of data and testing available to interested parties has potentially very large commercial and socially positive impacts. As discussed above, the complexity and high cost of using medical grade sensors to gather this type of information make it a long and difficult process.

In the following description, numerous specific details are introduced to provide a thorough understanding of, and enabling description for, embodiments of a sensor device, also referred to herein as an integrated headset, or an integrated sensor headset. One skilled in the relevant art, however, will recognize that these embodiments can be practiced without one or more of the specific details, or with other components, systems, etc. In other instances, well-known structures or operations are not shown, or are not described in detail, to avoid obscuring aspects of the disclosed embodiments.

A device is described that integrates sensors into a housing which can be placed on a human head for measurement of physiological data. The device includes at least one sensor and a reference electrode connected to the housing. A processor coupled to the sensor and the reference electrode receives signals that represent electrical activity in tissue of a user. The processor generates an output signal including data of a difference between an energy level in each of a first and second frequency band of the signals. The difference between energy levels is proportional to release level present time emotional state of the user. The device includes a wireless transmitter that transmits the output signal to a remote device. The device therefore processes the physiological data to create the output signal that correspond to a person's mental and emotional state (response).

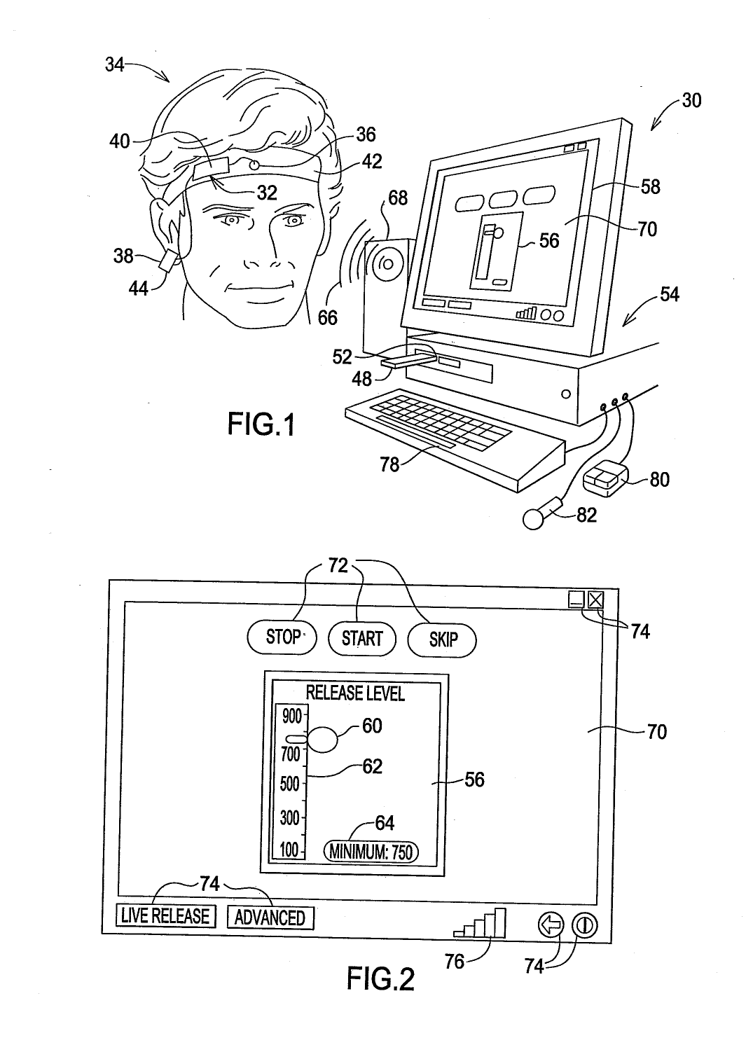

A system 30 which incorporates the present discussion is shown in FIG. 1. Exemplary system 30 includes a sensor device 32 which is connected to a user 34 for sensing and isolating a signal of interest from electrical activity in the user's pre-frontal lobe. The signal of interest has a measurable characteristic of electrical activity, or signal of interest, which relates to a present time emotional state (PTES) of user 34. PTES relates to the emotional state of the user at a given time. For instance, if the user is thinking about something that causes the user emotional distress, then the PTES is different than when the user is thinking about something which has a calming affect on the emotions of the user. In another example, when the user feels a limiting emotion regarding thoughts, then the PTES is different than when the user feels a state of release regarding those thoughts. Because of the relationship between the signal of interest and PTES, system 30 is able to determine a level of PTES experienced by user 34 by measuring the electrical activity and isolating a signal of interest from other electrical activity in the user's brain.

In the present example, sensor device 32 includes a sensor electrode 36 which is positioned at a first point and a reference electrode 38 which is positioned at a second point. The first and second points are placed in a spaced apart relationship while remaining in close proximity to one another. The points are preferably within about 8 inches of one another, and in one instance the points are about 4 inches apart. In the present example, sensor electrode 36 is positioned on the skin of the user's forehead and reference electrode 38 is connected to the user's ear. The reference electrode can also be attached to the user's forehead, which may include positioning the reference electrode over the ear of the user.

Sensor electrode 36 and reference electrode 38 are connected to an electronics module 40 of sensor device 32, which is positioned near the reference electrode 38 to that they are located substantially in the same noise environment. The electronics module 40 may be located at or above the temple of the user or in other locations where the electronics module 40 is in close proximity to the reference electrode 38. In the present example, a head band 42 or other mounting device holds sensor electrode 36 and electronics module 40 in place near the temple while a clip 44 holds reference electrode 38 to the user's ear. In one instance, the electronics module and reference electrode are positioned relative to one another such that they are capacitively coupled.

Sensor electrode 36 senses the electrical activity in the user's pre-frontal lobe and electronics module 40 isolates the signal of interest from the other electrical activity present and detected by the sensor electrode. Electronics module 40 includes a wireless transmitter 46, (FIG. 6), which transmits the signal of interest to a wireless receiver 48 over a wireless link 50. Wireless receiver 48, FIG. 1, receives the signal of interest from electronics module 40 and connects to a port 52 of a computer 54, or other device having a processor, with a port connector 53 to transfer the signal of interest from wireless receiver 48 to computer 54. Electronics module 40 includes an LED 55 (FIG. 6), and wireless receiver 48 includes an LED 57 which both illuminate when the wireless transmitter and the wireless receiver are powered.

In the present example, levels of PTES derived from the signal of interest are displayed in a meter 56, (FIGS. 1 and 2), on a computer screen 58 of computer 54. In this instance, computer 54, and screen 58 displaying meter 56 serve as an indicator. Levels of detail of meter 56 can be adjusted to suit the user. Viewing meter 56 allows user 34 to determine their level of PTES at any particular time in a manner which is objective. The objective feedback obtained from meter 56 is used for guiding the user to improve their PTES and to determine levels of PTES related to particular memories or thoughts which can be brought up in the mind of user 34 when the user is exposed to certain stimuli. Meter 56 includes an indicator 60 which moves vertically up and down a numbered bar 62 to indicated the level of the user's PTES. Meter 56 also includes a minimum level indicator 64 which indicates a minimum level of PTES achieved over a certain period of time or during a session in which user 34 is exposed to stimuli from media material 66. Meter 56 can also include the user's maximum, minimum and average levels of release during a session. Levels of PTES may also be audibly communicated to the user, and in this instance, the computer and speaker serve as the indicator. The levels can also be indicated to the user by printing them on paper.

In another instance, different release levels relating to reaction to the same media material can be stored over time on a memory device. These different release levels can be displayed next to one another to inform the user on his or her progress in releasing the negative emotions related to the media material.

In system 30, media material 66 is used to expose user 34 to stimuli designed to cause user 34 to bring up particular thoughts or emotions which are related to a high level of PTES in the user. In the present example, media material 66 includes audio material that is played though computer 54 over a speaker 68. Media material 66 and meter 56 are integrated into a computer program 70 which runs on computer 54 and is displayed on computer screen 58. Media material 66 is controlled using on-screen buttons 72, in this instance. Computer program 70 also has other menu buttons 74 for manipulation of program functions and an indicator 76 which indicates connection strength of the wireless link 50. Program 70 is typically stored in memory of computer 54, this or another memory device can also contain a database for storing self reported journals and self-observed progress.

In some instances, program 70 may require a response or other input from user 34. In these and other circumstances, user 34 may interact with program 70 using any one or more suitable peripheral or input device, such as a keyboard 78, mouse 80 and/or microphone 82. For instance, mouse 80 may be used to select one of buttons 72 for controlling media material 66.

Media material 66 allows user 34 to interact with computer 54 for self or assisted inquiry. Media material 66 can be audio, visual, audio and visual, and/or can include written material files or other types of files which are played on or presented by computer 54. Media material 66 can be based on one or more processes, such as “The Release Technique” or others. In some instances, generic topics can be provided in the form of audio-video files presented in the form of pre-described exercises. These exercises can involve typical significant life issues or goals for most individuals, such as money, winning, relationships, and many other popular topics that allow the user to achieve a freedom state regarding these topics. The freedom state about the goal can be displayed when a very low level of PTES, (under some preset threshold) is achieved by the user regarding the goal. The release technique is used as an example in some instances; other processes may also be used with the technological approach described herein.

In one instance, media material 66 involving “The Release Technique” causes user 34 to bring up a limiting emotion or an emotion-laden experience type of PTES, which results in a disturbance in the nervous system of the user. The process then guides user 34 to normalize the nervous system or release the emotion while the user is focused on the perceived cause of the disturbance. When it is determined that the level of PTES, or release level in this instance, is below a preset threshold then the process is completed.

The signal of interest which relates to the release level PTES are brain waves or electrical activity in the pre-frontal lobe of the user's brain in the range of 4-12 Hz. These characteristic frequencies of electrical activity are in the Alpha and Theta bands. Alpha band activity is in the 8 to 12 Hz range and Theta band activity is in the 4 to 7 Hz range. A linear relationship between amplitudes of the Alpha and Theta bands is an indication of the release level. When user 34 is in a non-release state, the activity is predominantly in the Theta band and the Alpha band is diminished; and when user 34 is in a release state the activity is predominantly in the Alpha band and the energy in the Theta band is diminished.

When user 34 releases the emotion, totality of thoughts that remain in the subconscious mind is lowered in the brain as the disturbance is incrementally released from the mind. A high number of thoughts in the subconscious mind results in what is known as unhappiness or melancholy feelings, which are disturbances in the nervous system. A low number of thoughts in the subconscious mind results in what is known as happiness or joyful feelings, which results in a normalization or absence of disturbances in the nervous system.

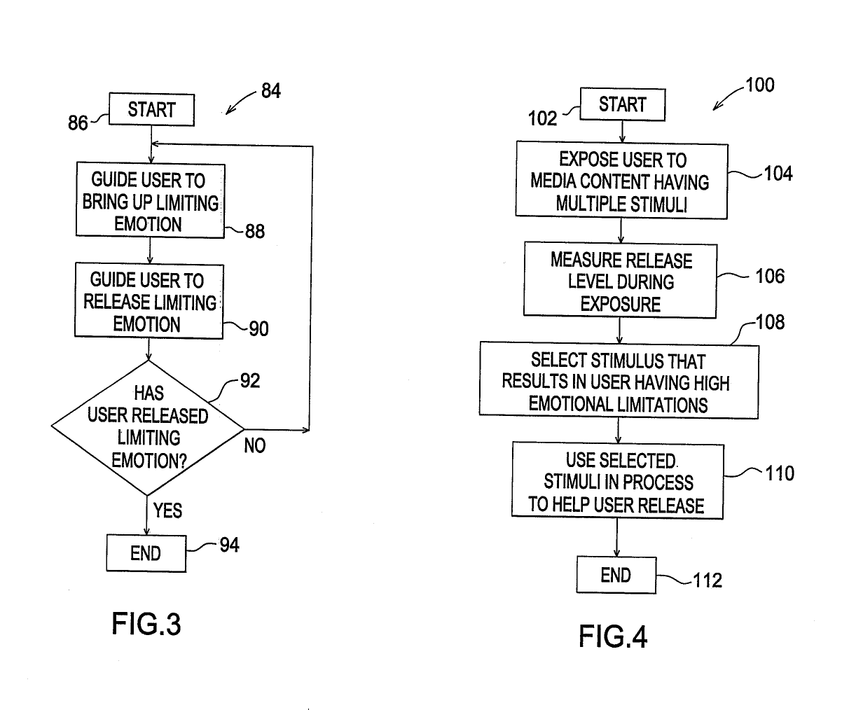

An exemplary method 84 which makes use of one or more self or assisted inquiry processes is shown in FIG. 3. Method 84 begins at a start 86 from which the method moves to a step 88. At step 88, program 70 uses stimuli in media material 66 to guide user 34 to bring up thoughts or subjects which causes an emotional disturbance in the PTES such as a limiting emotion. In the present example, media material 66 involves questions or statements directed to user 34 through speaker 68. In this and other instances, the computer can insert statements about goals or issue which were input by the user into the media material 66. For example, user 34 may input a goal statement using keyboard 78 and the computer may generate a voice which inserts the goal statement into the media material. In another example, the user may input the goal statement using microphone 82 and the computer may insert the goal statement into the media material.

Method 84 then proceeds to step 90 where program 70 uses media material 66 to guide user 34 to release the liming emotions while still focusing on the thought or subject which causes the limiting emotion. From step 90, the program proceeds to step 92 where a determination is made as to whether user 34 has released the limiting emotions. This determination is made using the signal of interest from sensor device 32. In the instance case, the level of release is indicated by the position of indicator 60 on bar 62 in meter 56, as shown in FIG. 2. If the meter indicates that user 34 has released the limiting emotions to an appropriate degree, such as below the preset threshold, then the determination at 92 is yes and method 84 proceeds to end at step 94. If the determination at 92 is that user 34 has not release the limiting emotions to an appropriate degree, then the determination at 92 is no, and method 84 returns to step 88 to again guide the user to bring up the thought or subject causing the limiting emotion. Method 84 can be continued as long as needed for user 34 to release the limiting emotions and achieve the freedom state. Processes can also include clean up sessions in which the user is guided by the media material to release many typical limiting emotions to assist the user in achieving a low thought frequency releasing the limiting emotions.

By observing meter 56 while attempting to release the limiting emotions, user 34 is able to correlate feelings with the release of limiting emotions. Repeating this process reinforces the correlation so that the user learns what it feels like to release and is able to release effectively with or without the meter 56 by having an increased releasing skill. A loop feature allows the user to click on a button to enter a loop session in which the releasing part of an exercise is repeated continuously. The levels of the user's PTES are indicated to the user and the levels are automatically recorded during these loop sessions for later review. Loop sessions provide a fast way in which to guide a user to let go of limiting emotions surrounding particular thoughts related to particular subjects. The loop session does not require the user to do anything between repetitions which allows them to maintain the desirable state of low thought activity, or the release state. Loop sessions can be included in any process for guiding the user to improve their PTES.

Computer 54 is also able to record release levels over time to a memory device to enable user 34 to review the releasing progress achieved during a recorded session. Other sessions can be reviewed along side of more recent sessions to illustrate the progress of the user's releasing ability by recalling the sessions from the memory device.

System 30 is also used for helping user 34 to determine what particular thoughts or subjects affect the user's PTES. An example of this use is a method 100, shown in FIG. 4. Method 100 begins at start 102 from which the method proceeds to step 104. At step 104, user 34 is exposed to a session of media content 42 which contains multiple stimuli that are presented to user 34 over time. Method 100 proceeds to step 106 where the levels of PTES of user 34 are determined during the session while the user is exposed to the multiple stimuli. Following step 106 method proceeds to step 108 where stimulus is selected from the media content 42 which resulted in negative affects on the PTES, such as high emotional limitations. Method 100 therefore identifies for the user areas which results in the negative affects on the PTES. Method 100 then proceeds to step 110 where the selected stimuli is used in a process to help the user release the negative emotions. Method 100 ends at step 112.

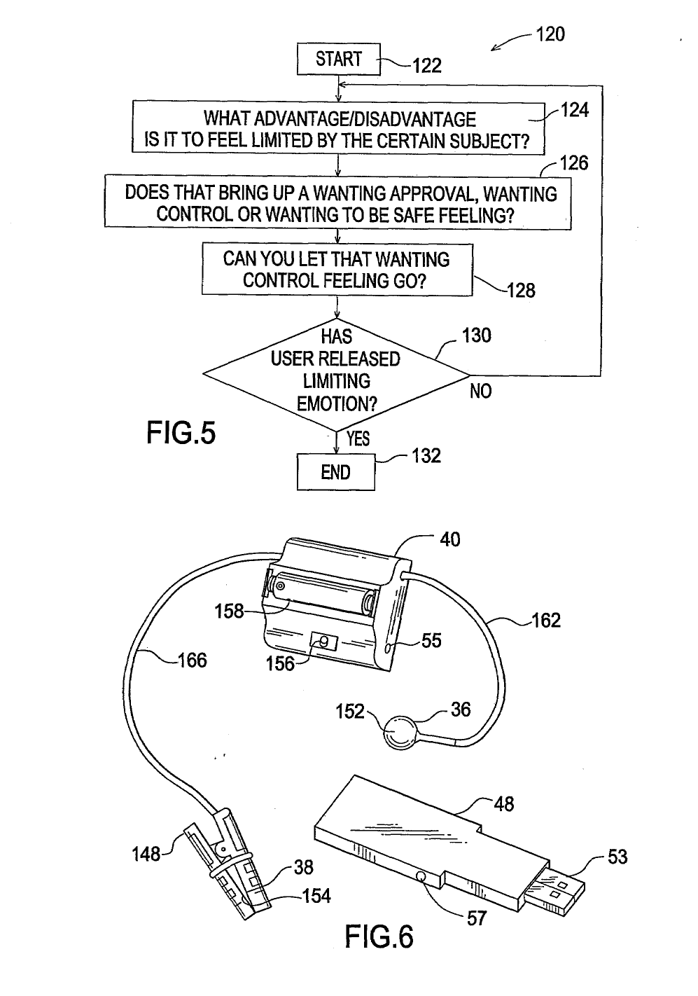

In one example, program 70 uses a method 120, FIG. 5, which includes a questioning pattern called “Advantages/Disadvantages.” In this method, the media file asks user 34 several questions in sequence related to advantages/disadvantages of a “certain subject”, which causes the user to experience negative emotions. Words or phrases of the “certain subject” can be entered into the computer by the user using one of the input devices, such as keyboard 78, mouse 80 and/or microphone 82 which allows the computer to insert the words or phrases into the questions. System 30 may also have goal documents that have the user's goal statements displayed along with the questioning patterns about the goal and release level data of the user regarding the goal. As an example, the user may have an issue which relates to control, such as a fear of being late for an airline flight. In this instance, the user would enter something like “fear of being late for a flight” as the “certain subject.”

Series of questions related to advantages and disadvantage can be alternated until the state of release, or other PTES, is stabilized as low as possible, that is with the greatest amount of release. Method 120, shown in FIG. 5, starts at a start 122 from which it proceeds to step 124 where program 70 asks user 34 “What advantage/disadvantage is it to me to feel limited by the certain subject?” Program 70 then waits for feedback from the user through one of the input devices.

Program then proceeds to step 126 where program 70 asks user 34 “Does that bring up a wanting approval, wanting control or wanting to be safe feeling?” Program 70 waits for a response from user 34 from the input device and deciphers which one of the feelings the user responds with, such as “control feeling” for instance. Method 120 then proceeds to step 128 where program 70 questions the user based on the response given to step 128 by asking “Can you let that wanting control feeling go?” in this instance. At this point method 120 proceeds to step 130 where sensor device 32 determines the signal of interest to determine the release level of user 34. The release level is monitored and the media file stops playing when the release level has stabilized at its lowest point. At this time method 120 proceeds to step 132 and the session is complete. When the session is complete, user 34 will feel a sense of freedom regarding the certain subject. If some unwanted emotional residue is left, this same process can be repeated until complete freedom regarding the issue is realized by the user.

The above method is an example of “polarity releasing” in which an individual is guided to think about positives and negatives about a certain subject or particular issue, until the mind gives up on the negative emotions generated by the thoughts. There are other polarity releasing methods, such as “Likes/Dislikes” and other concepts and methods that help user's to achieve lower though frequency which may also be used along with a sensor device such as sensor device 32 for the purposes described herein.

Program 70 can store the history of responses to media on a memory device, and combine multiple iterations of responses to the same media in order to create a chart of improvement for user 34. Plotting these responses on the same chart using varying colors and dimensional effects demonstrates to user 34 the various PTES reactions over time to the same media stimulus, demonstrating improvement.

Program 70 can store reaction to live content as well. Live content can consist of listening to a person or audio in the same physical location, or listening to audio streaming over a telecommunications medium like telephone or the Internet, or text communications. Program 70 can send the PTES data from point-to-point using a communication medium like the Internet. With live content flowing in one direction, and PTES data flowing in the other, the deliverer of live content has a powerful new ability to react and change the content immediately, depending on the PTES data reaction of the individual. This deliverer may be a person or a web server application with the ability to understand and react to changing PTES.

Program 70 can detect the version of the electronic module 40 latently, based on the type of data and number of bytes being sent. This information is used to turn on and off various features in the program 70, depending on the feature's availability in the electronic module 40.

With certain types of computers and when certain types of wireless links are used, an incompatibility between wireless receiver 48 and computer 54 may occur. This incompatibility between an open host controller interface (OHCI) of the computer 54 and a universal host controller interface (UHCI) chip in the wireless receiver 48 causes a failure of communication. Program 70 has an ability to detect the symptom of this specific incompatibility and report it to the user. The detection scheme looks for a single response to a ping ‘P’ from the wireless receiver 48, and all future responses to a ping are ignored. Program 70 then displays a modal warning to the user suggesting workarounds for the incompatibility.

Program 70 detects the disconnecting of wireless link 50 by continually checking for the arrival of new data. If new data stops coming in, it assumes a wireless link failure, and automatically pauses the media being played and recording of PTES data. On detection of new data coming into the computer 54, the program 70 automatically resumes the media and recording.

Program 70 can create exercises and set goals for specific PTES levels. For example, it asks the user to set a target level of PTES and continues indefinitely until the user has reached that goal. Program 70 can also store reactions during numerous other activities. These other activities include but are not limited to telephone conversations, meetings, chores, meditation, and organizing. In addition, program 70 can allow users to customize their sessions by selecting audio, title, and length of session.

Other computing devices, which can include processor based computing devices, (not shown) can be used with sensor device 32 to play media material 66 and display or otherwise indicate the PTES. These devices may be connected to the sensor device 32 utilizing an integrated wireless receiver rather than the separate wireless receiver 48 which plugs into the port of the computer. These devices are more portable than computer 54 which allows the user to monitor the level PTES throughout the day or night which allows the user to liberate the subconscious mind more rapidly. These computing devices can include a camera with an audio recorder for storing and transmitting data to the receiver to store incidents of reactivity on a memory device for review at a later time. These computing devices can also upload reactivity incidents, intensity of these incidents and/or audio-video recordings of these incidents into computer 54 where the Attachment and Aversions process or other process can be used to permanently reduce or eliminate reactivity regarding these incidents.

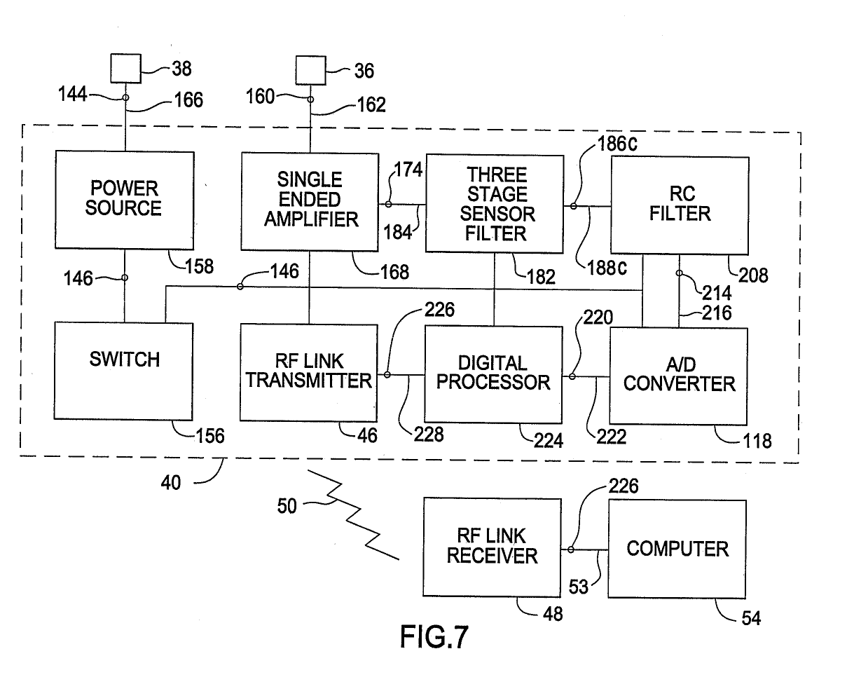

One example of sensor device 32 is shown in FIGS. 6 and 7. Sensor device 32 includes sensor electrode 36, reference electrode 38 and electronics module 40. The electronics module 40 amplifies the signal of interest by 1,000 to 100,000 times while at the same time insuring that 60 Hz noise is not amplified at any point. Electronics module 40 isolates the signal of interest from undesired electrical activity.

Sensor device 32 in the present example also includes wireless receiver 48 which receives the signal of interest from the electronics module over wireless link 50 and communicates the signal of interest to computer 54. In the present example, wireless link 50 uses radiofrequency energy; however other wireless technologies may also be used, such as infrared. Using a wireless connection eliminates the need for wires to be connected between the sensor device 32 and computer 54 which electrically isolates sensor device 32 from computer 54.

Reference electrode 38 is connected to a clip 148 which is used for attaching reference electrode 38 to an ear 150 of user 34, in the present example. Sensor electrode 36 includes a snap or other spring loaded device for attaching sensor electrode 36 to headband 42. Headband 42 also includes a pocket for housing electronics module 40 at a position at the user's temple. Headband 42 is one example of an elastic band which is used for holding the sensor electrode and/or the electronics module 40, another types of elastic bands which provide the same function could also be used, including having the elastic band form a portion of a hat.

Other types of mounting devices, in addition to the elastic bands, can also be used for holding the sensor electrode against the skin of the user. A holding force holding the sensor electrode against the skin of the user can be in the range of 1 to 4 oz. The holding force can be, for instance, 1.5 oz.

In another example of a mounting device involves a frame that is similar to an eyeglass frame, which holds the sensor electrode against the skin of the user. The frame can also be used for supporting electronics module 40. The frame is worn by user 34 in a way which is supported by the ears and bridge of the nose of the user, where the sensor electrode 36 contacts the skin of the user.

Sensor electrode 36 and reference electrode 38 include conductive surface 152 and 154, respectively, that are used for placing in contact with the skin of the user at points where the measurements are to be made. In the present example, the conductive surfaces are composed of a non-reactive material, such as copper, gold, conductive rubber or conductive plastic. Conductive surface 152 of sensor electrode 36 may have a surface area of approximately ½ square inch. The conductive surfaces 152 are used to directly contact the skin of the user without having to specially prepare the skin and without having to use a substance to reduce a contact resistance found between the skin and the conductive surfaces.

Sensor device 32 works with contact resistances as high as 500,000 ohms which allows the device to work with conductive surfaces in direct contact with skin that is not specially prepared. In contrast, special skin preparation and conductive gels or other substances are used with prior EEG electrodes to reduce the contact resistances to around 20,000 ohms or less. One consequence of dealing with higher contact resistance is that noise may be coupled into the measurement. The noise comes from lights and other equipment connected to 60 Hz power, and also from friction of any object moving through the air which creates static electricity. The amplitude of the noise is proportional to the distance between the electronics module 40 and the reference electrode 38. In the present example, by placing the electronics module over the temple area, right above the ear and connecting the reference electrode to the ear, the sensor device 32 does not pick up the noise, or is substantially unaffected by the noise. By positioning the electronics module in the same physical space with the reference electrode and capacitively coupling the electronics module with the reference electrode ensures that a local reference potential 144 in the electronics module and the ear are practically identical in potential. Reference electrode 38 is electrically connected to local reference potential 144 used in a power source 158 for the sensor device 32.

Power source 158 provides power 146 to electronic components in the module over power conductors. Power source 158 provides the sensor device 32 with reference potential 144 at 0 volts as well as positive and negative source voltages, −VCC and +VCC. Power source 158 makes use of a charge pump for generating the source voltages at a level which is suitable for the electronics module.

Power source is connected to the other components in the module 40 though a switch 156. Power source 158 can include a timer circuit which causes electronics module 40 to be powered for a certain time before power is disconnected. This feature conserves power for instances where user 34 accidentally leaves the power to electronics module 40 turned on. The power 146 is referenced locally to measurements and does not have any reference connection to an external ground system since sensor circuit 32 uses wireless link 50.

Sensor electrode 36 is placed in contact with the skin of the user at a point where the electrical activity in the brain is to be sensed or measured. Reference electrode 38 is placed in contact with the skin at a point a small distance away from the point where the sensor electrode is placed. In the present example, this distance is 4 inches, although the distance may be as much as about 8 inches. Longer lengths may add noise to the system since the amplitude of the noise is proportional to the distance between the electronics module and the reference electrode. Electronics module 40 is placed in close proximity to the reference electrode 38. This causes the electronics module 40 to be in the same of electrical and magnetic environment is the reference electrode 38 and electronics module 40 is connected capacitively and through mutual inductance to reference electrode 38. Reference electrode 38 and amplifier 168 are coupled together into the noise environment, and sensor electrode 36 measures the signal of interest a short distance away from the reference electrode to reduce or eliminate the influence of noise on sensor device 32. Reference electrode 38 is connected to the 0V in the power source 158 with a conductor 166.

Sensor electrode 36 senses electrical activity in the user's brain and generates a voltage signal 160 related thereto which is the potential of the electrical activity at the point where the sensor electrode 36 contacts the user's skin relative to the local reference potential 144. Voltage signal 160 is communicated from the electrode 36 to electronics module 40 over conductor 162. Conductors 162 and 166 are connected to electrodes 36 and 38 in such a way that there is no solder on conductive surfaces 152 and 154. Conductor 162 is as short as practical, and in the present example is approximately 3 inches long. When sensor device 32 is used, conductor 162 is held a distance away from user 34 so that conductor 162 does not couple signals to or from user 34. In the present example, conductor 162 is held at a distance of approximately ½″ from user 34. No other wires, optical fibers or other types of extensions extend from the electronics module 40, other than the conductors 162 and 166 extending between module 40 and electrodes 36 and 38, since these types of structure tend to pick up electronic noise.

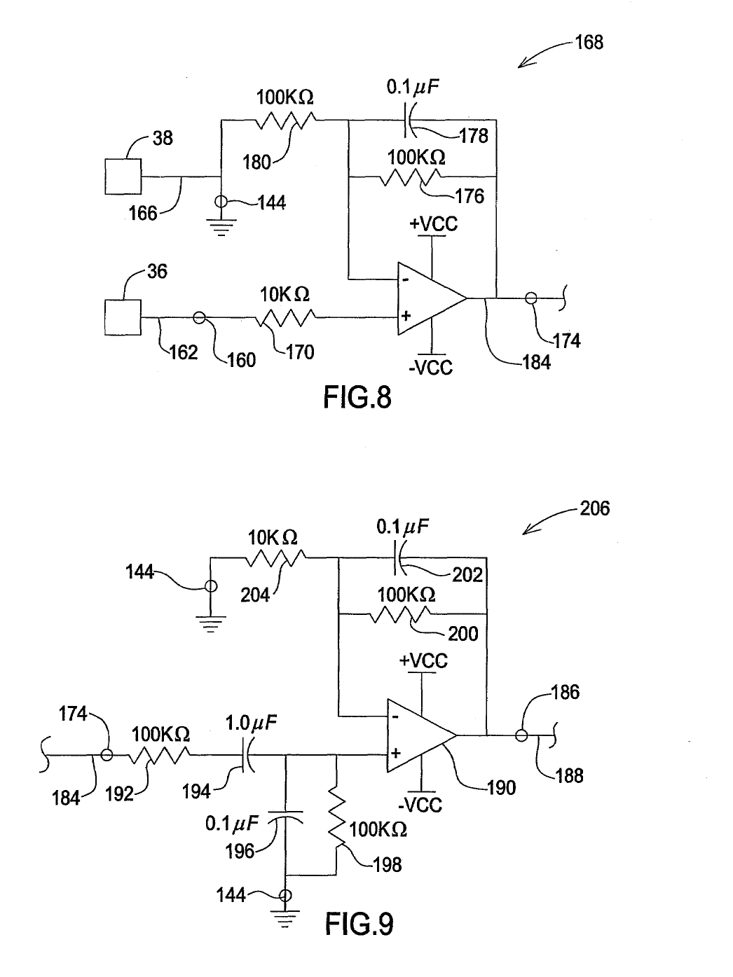

The electronics module 40 measures or determines electrical activity, which includes the signal of interest and other electrical activity unrelated to the signal of interest which is undesired. Electronics module 40 uses a single ended amplifier 168, (FIGS. 7 and 8), which is closely coupled to noise in the environment of the measurement with the reference electrode 38. The single ended amplifier 168 provides a gain of 2 for frequencies up to 12 Hz, which includes electrical activity in the Alpha and Theta bands, and a gain of less than 1 for frequencies 60 Hz and above, including harmonics of 60 Hz.

Amplifier 168, FIGS. 8 and 11, receives the voltage signal 160 from electrode 36 and power 146 from power source 158. Single ended amplifier 168 generates an output signal 174 which is proportional to voltage signal 160. Output signal 174 contains the signal of interest. In the present example, voltage signal 160 is supplied on conductor 162 to a resistor 170 which is connected to non-inverting input of high impedance, low power op amp 172. Output signal 174 is used as feedback to the inverting input of op amp 172 through resistor 176 and capacitor 178 which are connected in parallel. The inverting input of op amp 172 is also connected to reference voltage 144 through a resistor 180.

Amplifier 168 is connected to a three-stage sensor filter 182 with an output conductor 184 which carries output signal 174. The electrical activity or voltage signal 160 is amplified by each of the stages 168 and 182 while undesired signals, such as those 60 Hz and above, are attenuated by each of the stages. Three-stage sensor filter has three stages 206a, 206b and 206c each having the same design to provide a bandpass filter function which allows signals between 1.2 and 12 Hz to pass with a gain of 5 while attenuating signal lower and higher than these frequencies. The bandpass filter function allows signals in the Alpha and Theta bands to pass while attenuating noise such as 60 Hz and harmonics of the 60 Hz. The three stage sensor filter 182 removes offsets in the signal that are due to biases and offsets in the parts. Each of the three stages is connected to source voltage 146 and reference voltage 144. Each of the three stages generates an output signal 186a, 186b and 186c on an output conductor 188a, 186b and 188c, respectively.

In the first stage 206a, FIGS. 9 and 11, of three-stage sensor filter 182, output signal 174 is supplied to a non-inverting input of a first stage op-amp 190a through a resistor 192a and capacitor 194a. A capacitor 196a and another resistor 198a are connected between the non-inverting input and reference voltage 144. Feedback of the output signal 186a from the first stage is connected to the inverting input of op amp 190a through a resistor 200a and a capacitor 202a which are connected in parallel. The inverting input of op amp 190a is also connected to reference voltage 144 through resistor 204a.

Second and third stages 206b and 206c, respectively, are arranged in series with first stage 206a. First stage output signal 186a is supplied to second stage 206b through resistor 192b and capacitor 194b to the non-inverting input of op-amp 190b. Second stage output signal 186b is supplied to third stage 206c through resistor 192c and capacitor 194c. Resistor 198b and capacitor 196b are connected between the non-inverting input of op-amp 190b and reference potential 144, and resistor 198c and capacitor 196c are connected between the non-inverting input of op-amp 190c and reference potential 144. Feedback from output conductor 188b to the inverting input of op-amp 190b is through resistor 200b and capacitor 202b and the inverting input of op-amp 190b is also connected to reference potential 144 with resistor 204b. Feedback from output conductor 188c to the inverting input of op-amp 190c is through resistor 200c and capacitor 202c and the inverting input of op-amp 190c is also connected to reference potential 144 with resistor 204c.

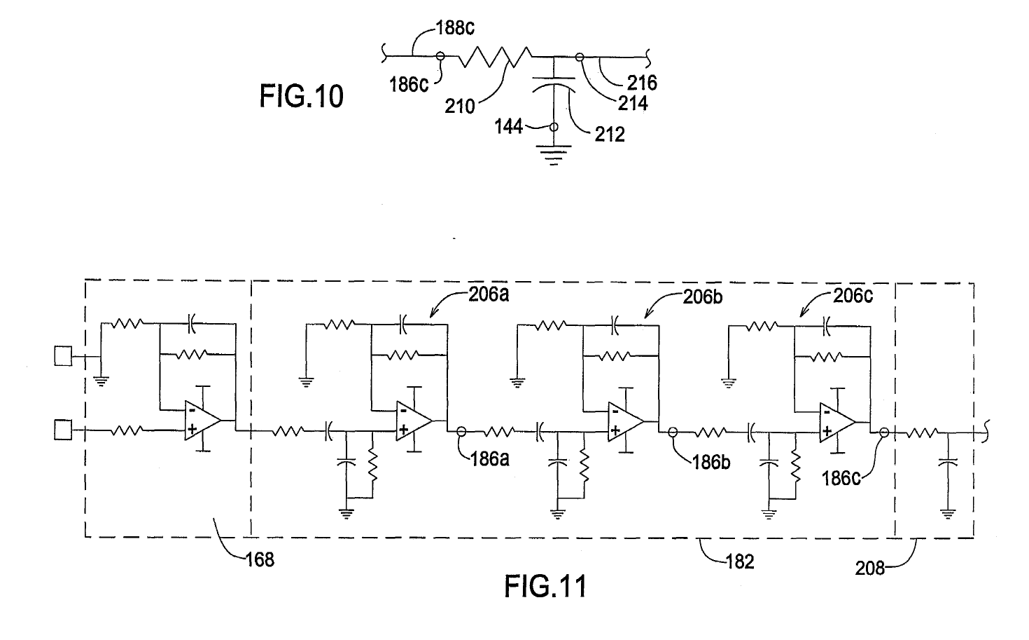

Three stage sensor filter 182 is connected to an RC filter 208, FIGS. 10 and 11, with the output conductor 188c which carries the output signal 186c from third stage 206c of three stage sensor filter 182, FIG. 7. RC filter 208 includes a resistor 210 which is connected in series to an output conductor 216, and a capacitor 212 which connects between reference potential 144 and output conductor 216. RC filter serves as a low pass filter to further filter out frequencies above 12 Hz. RC filter 208 produces a filter signal 214 on output conductor 216. RC filter 208 is connected to an analog to digital (A/D) converter 218, FIG. 7.

A/D converter 218 converts the analog filter signal 214 from the RC filter to a digital signal 220 by sampling the analog filter signal 214 at a sample rate that is a multiple of 60 Hz. In the present example the sample rate is 9600 samples per second. Digital signal 220 is carried to a digital processor 224 on an output conductor 222.

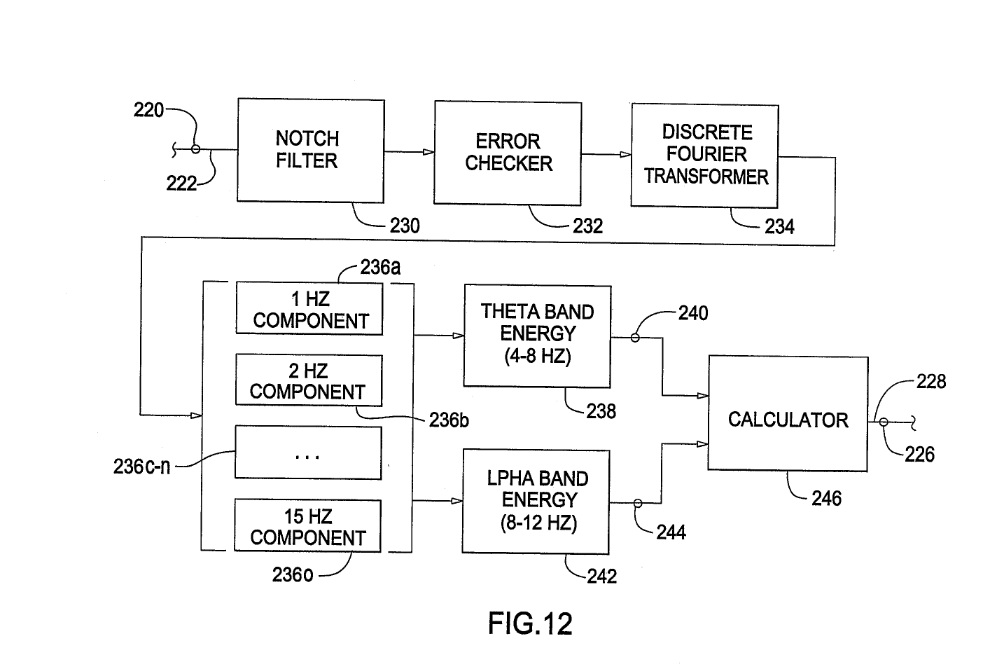

Digital processor 224, FIGS. 7 and 12 provides additional gain, removal of 60 Hz noise, and attenuation of high frequency data. Digital processor 224 many be implemented in software operating on a computing device. Digital processor 224 includes a notch filter 230, FIG. 12 which sums 160 data points of digital signal 220 at a time to produce a 60 Hz data stream that is free from any information at 60 Hz. Following notch filter 230 is an error checker 232. Error checker 232, removes data points that are out of range from the 60 Hz data stream. These out of range data points are either erroneous data or they are cause by some external source other than brain activity.

After error checker 232, digital processor 224 transforms the data stream using a discreet Fourier transformer 234. While prior EEG systems use band pass filters to select out the Alpha and Theta frequencies, among others, these filters are limited to processing and selecting out continuous periodic functions. By using a Fourier transform, digital processor 224 is able to identify randomly spaced events. Each event has energy in all frequencies, but shorter events will have more energy in higher frequencies and longer events will have more energy in lower frequencies. By looking at the difference between the energy in Alpha and Theta frequencies, the system is able to identify the predominance of longer or shorter events. The difference is then scaled by the total energy in the bands. This causes the output to be based on the type of energy and removes anything tied to amount of energy.

The Fourier transformer 234 creates a spectrum signal that separates the energy into bins 236a to 236o which each have a different width of frequency. In one example, the spectrum signal has 30 samples and separates the energy spectrum into 2 Hz wide bins; in another example, the spectrum signal has 60 samples and separates the bins into 1 Hz wide bins. Bins 236 are added to create energy signals in certain bands. In the present example, bins 236 between 4 and 8 Hz are passed to a summer 238 which sums these bins to create a Theta band energy signal 240; and bins between 8 and 12 Hz are passed to a summer 242 which sums these bins to create an Alpha band energy signal 244.

In the present example, the Alpha and Theta band energy signals 240 and 244 passed to a calculator 246 which calculates (Theta−Alpha)/Theta+Alpha) and produces an output signal 226 on a conductor 228 as a result.

Output signal 226, FIG. 7, is passed to wireless transmitter 46 which transmits the output signal 226 to wireless receiver 48 over wireless link 50. In the present example, output signal 226 is the signal of interest which is passed to computer 54 through port 52 and which is used by the computer to produce the PTES for display in meter 56.

Computer 54 may provide additional processing of output signal 226 in some instances. In the example using the Release Technique, the computer 54 manipulates output signal 226 to determine relative amounts of Alpha and Theta band signals in the output signal to determine levels of release experienced by user 34.

A sensor device utilizing the above described principles and feature can be used for determining electrical activity in other tissue of the user in addition to the brain tissue just described, such as electrical activity in muscle and heart tissue. In these instances, the sensor electrode is positioned on the skin at the point where the electrical activity is to be measured and the reference electrode and electronics module are positioned nearby with the reference electrode attached to a point near the sensor electrode. The electronics module, in these instances, includes amplification and filtering to isolate the frequencies of the muscle or heart electrical activity while filtering out other frequencies.

There are many practical applications of physiological data that could be enabled with a non-intrusive sensing device (sensor) that allows a test subject to participate in normal activities with a minimal amount of interference from the device, as described above. The data quality of this device need not be as stringent as a medical device as long as the device measures data accurately enough to satisfy the needs of parties interested in such data, making it possible to greatly simplify the use and collection of physiological data when one is not concerned about treating any disease or illness. There are various types of non-intrusive sensors that are in existence. For a non-limiting example, modern three axis accelerometer can exist on a single silicon chip and can be included in many modern devices. The accelerometer allows for tracking and recording the movement of whatever subject the accelerometer is attached to. For another non-limiting example, temperature sensors have also existed for a long time in many forms, with either wired or wireless connections. All of these sensors can provide useful feedback about a test subject's responses to stimuli, but thus far, no single device has been able to incorporate all of them seamlessly. Attaching each of these sensors to an individual separately is timing consuming and difficult, requiring a trained professional to insure correct installation and use. In addition, each newly-added sensor introduces an extra level of complexity, user confusion, and bulk to the testing instrumentation.

As described above an integrated headset is introduced, which integrates a plurality of sensors into one single piece and can be placed on a person's head for measurement of his/her physiological data. Such integrated headset is adaptive, which allows adjustability to fit the specific shape and/or size of the person's head. The integrated headset minimizes data artifacts arising from at least one or more of: electronic interference among the plurality of sensors, poor contacts between the plurality of sensors and head movement of the person. In addition, combining several types of physiological sensors into one piece renders the measured physiological data more robust and accurate as a whole.

The integrated headset of an embodiment integrates a plurality of sensors into one single piece and can be placed on a person's head for measurement of his/her physiological data. Such integrated headset is easy to use, which measures the physiological data from the person accurately without requiring any conductive gel or skin preparation at contact points between the plurality of sensors and the person's skin. In addition, combining several types of physiological sensors into one piece renders the measured physiological data more robust and accurate as a whole.

The integrated headset of an embodiment integrates a plurality of sensors into one single piece and can be placed on a person's head for measurement of his/her physiological data. Such integrated headset is non-intrusive, which allows the person wearing the headset to freely conduct a plurality of functions without any substantial interference from the physiological sensors integrated in the headset. In addition, combining several types of physiological sensors into one piece renders the measured physiological data more robust and accurate as a whole.

Having a single device that incorporates numerous sensors also provides a huge value for advertisers, media producers, educators and many other parties interested in physiological data. These parties desire to understand the reactions and responses people have to their particular stimulus in order to tailor their information or media to better suit the needs of end users and/or to increase the effectiveness of the media. By sensing these exact changes instead of using focus groups, surveys, knobs or other easily biased measures of response, the integrated sensor improves both the data that is measured and recorded and the granularity of such data, as physiological data can be recorded by a computer program/device many times per second. The physiological data can also be mathematically combined from the plurality of sensors to create specific outputs that corresponds to a person's mental and emotional state (response).

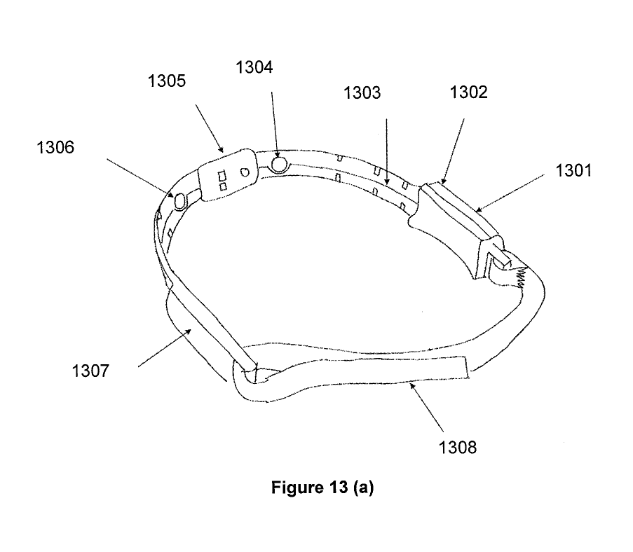

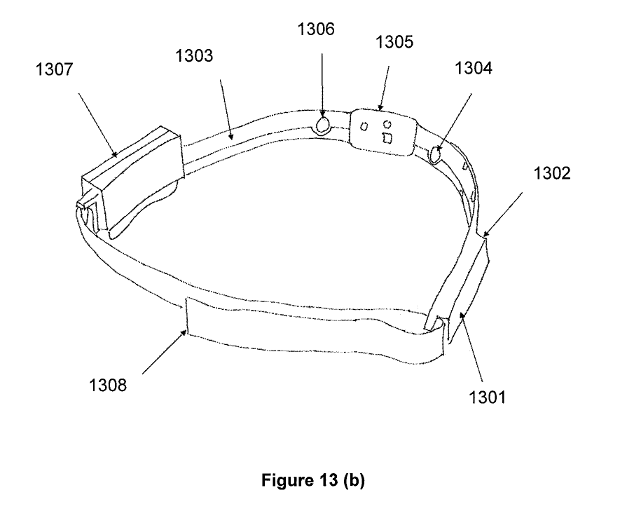

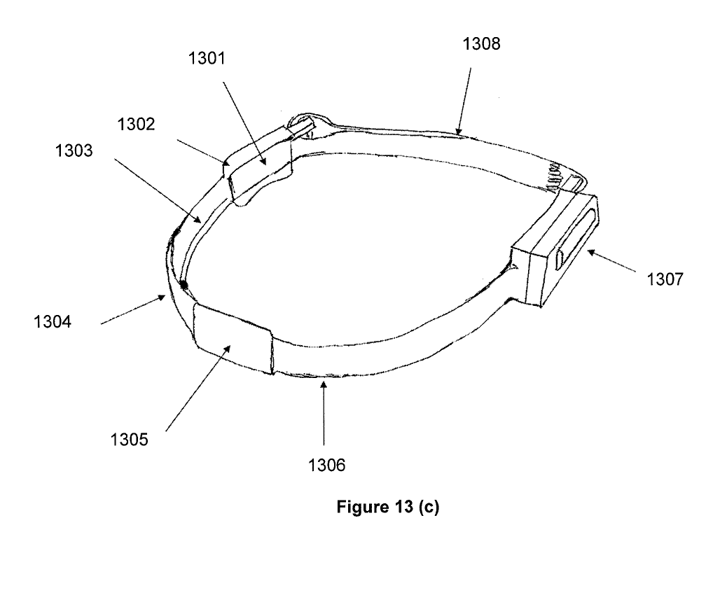

As a more specific example embodiment of the sensor headset described above, FIGS. 13a-13c show several views of a sensor headset, under an embodiment. Although the diagrams depict components as functionally separate, such depiction is merely for illustrative purposes. It will be apparent to those skilled in the art that the components portrayed in this figure can be arbitrarily combined or divided into separate software, firmware and/or hardware components. Furthermore, it will also be apparent to those skilled in the art that such components, regardless of how they are combined or divided, can execute on the same computing device or multiple computing devices, and wherein the multiple computing devices can be connected by one or more networks.

Referring to FIGS. 13(a)-(c), the integrated headset may include at least one or more of the following components: a processing unit 1301, which can be but is not limited to a microprocessor, functions as a signal collection, processing and transmitting circuitry that collects, digitizes, and processes the physiological data measured from a person who wears the headset and transmits such data to a separate/remote location. A motion detection unit 1302, which can be but is not limited to a three axis accelerometer, senses movement of the head of the person. A stabilizing component 1303, which can be but is not limited to a silicon stabilization strip, stabilizes and connects the various components of the headset together. Such stabilizing component provides adhesion to the head by surface tension created by a sweat layer under the strip to stabilize the headset for more robust sensing through stabilization of the headset that minimizes responses to head movement of the person. A set of EEG electrodes, which can be but is not limited to a right EEG electrode 1304 and a left EEG electrode 1306 positioned symmetrically about the centerline of the forehead of the person, can be utilized to sense/measure EEG signals from the person. The electrodes may also have another contact on one ear of the person for a ground reference. These EEG electrodes can be prefrontal dry electrodes that do not need conductive gel or skin preparation to be used, where contacts are needed between the electrodes and the skin of the person but without excessive pressure applied. A heart rate sensor 1305 is a robust blood volume pulse sensor that can measure the person's heart rate and the sensor can be positioned directly in the center of the forehead of the person between the set of EEG electrodes. A power handling and transmission circuitry 1307, which can be but is not limited to a rechargeable or replaceable battery module, can provide operating power to the components of the headset and can be located over one of the person's ears. An adjustable strap 1308 positioned in the rear of the person's head can be used to adjust the headset to a comfortable tension setting for the shape and size of the person so that the pressure applied to the plurality of sensors is adequate for robust sensing without causing discomfort. Note that although motion detection unit, EEG electrodes, and heart rate sensor are used here as non-limiting examples of sensors, other types of sensors can also be integrated into the headset, wherein these types of sensors can be but are not limited to, electroencephalograms, blood oxygen sensors, galvanometers, electromygraphs, skin temperature sensors, breathing sensors, and any other types of physiological sensors.

In some embodiments, the integrated headset can be turned on with a push button and the test subject's physiological data can be measured and recorded instantly. Data transmission from the headset can be handled wirelessly through a computer interface to which the headset links. No skin preparation or conductive gels are needed on the tester to obtain an accurate measurement, and the headset can be removed from the tester easily and be instantly used by another person. No degradation of the headset occurs during use and the headset can be reused thousands of times, allowing measurement to be done on many participants in a short amount of time and at low cost.

In some embodiments, the accelerometer 1302 can be incorporated into an electronic package in a manner that allows its three axes to align closely to the regularly accepted axes directions in a three-dimensional space. Such requirement is necessary for the accelerometer to output data that can be easily interpreted without the need for complex mathematical operations to normalize the data to fit the standard three-axis system. Other sensors such as temperature sensors have less stringent location requirements and are more robust, which can be placed at various locations on the headset.

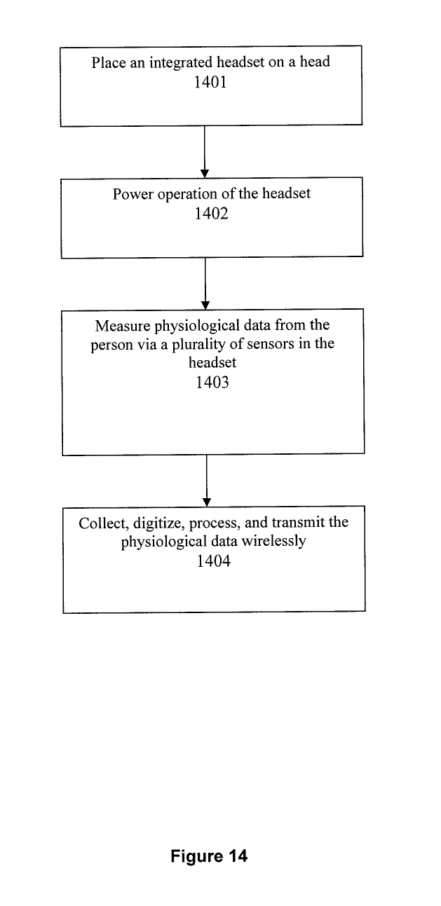

FIG. 14 is a flow chart illustrating an exemplary process to support measuring physiological data via an integrated headset in accordance with one embodiment of the present invention. Although this figure depicts functional steps in a particular order for purposes of illustration, the process is not limited to any particular order or arrangement of steps. One skilled in the art will appreciate that the various steps portrayed in this figure could be omitted, rearranged, combined and/or adapted in various ways.

Referring to FIG. 14, an integrated headset can be placed on a person's head at 1401, wherein the headset adjusts automatically to fit shape and/or size of the person's head. Operation of the headset can be powered via a powering unit at 1402. At 1403, a plurality of sensors in the headset can be utilized to measure physiological data from the person wearing the headset while allowing the person to freely conduct a plurality of functions without substantial interference from the plurality of sensors. Here, such functions include but are not limited to, watching a plurality of media instances or conducting his/her normal activities. Such measurement requires no conductive gel or skin preparation at contact points between the plurality of sensors and the person's skin.

At 1404, the physiological data can be collected, digitized, processed, and transmitted wirelessly with minimum artifacts via a signal processing unit in the headset. The signal processing unit minimizes data artifacts arising from at least one or more of: electronic interference among the plurality of sensors, poor contact between the plurality of sensors and the person's head, and head movement of the person.

The physiological signals emanating from a human being are extremely small, especially in comparison to the general environmental background noise that is always present. This presents a challenge for creating an integrated headset that is very stable and minimizes data artifacts, wherein the artifacts may arise from at least one or more of: electronic interference, poor contact points, head movement that creates static electricity.

One of the major problems in recording human physiological signals is the issue of electrical interference, which may come from either external environmental sources or the various sensors that are incorporated into the single headset, or both. Combining multiple sensors into a single integrated headset may cause electrical interference to leak from one component (sensor) over into another due to the very weak signals that are being detected. For a non-limiting example, an EEG electrode is very sensitive to interference and signals from other sensors can create artifacts in the EEG reading.

In some embodiments, data transmission from the headset can be handled wirelessly through a computer interface that the headset links to. Since wireless communication happens at high frequencies, the typical 50/60 Hz electrical noise that may, for a non-limiting example, be coupled to a signal wire and interfere with the measured data transferred by the wire can be minimized.

In some embodiments, power levels of one or more of the sensors integrated in the integrated headset may be tuned as low as possible to minimize the electrical interference. In addition, specific distance between signal-carrying wires of the sensors can also be set and enforced to reduce the (electronic) crosstalk between the wires.

In some embodiments, with reference to FIGS. 13(a)-(c), the power handling and transmission circuitry 1307 of the integrated headset can be separated from the signal collection and processing circuitry 1301. Being a wireless device, the integrated headset uses a battery and the noise generated by the battery may ruin the measurement as the battery noise is far larger than the electrical signals being measured. By physically separating the circuits and only delivering power by means of minimum number of wires needed, the integrated headset can cut down electrical interference significantly.

In some embodiments, the power and signal processing circuitry can be placed over opposite ears of the tester, respectively. A flat cable can be used to transmit the power from the battery module 1307 over the left ear to the signal processing circuitry 1301 over the right ear. The data from the heart rate sensor 1305 can also be carried using a similar flat cable, which allows greater control over wire placement and restricts the wires from moving around during use as in the case with conventional stranded wires. In addition, the EEG electrodes 1304 and 1306 can be wired using conventional stranded copper wire to carry the signal to the signal processing circuit 1301. The wires from the EEG electrodes can be placed at the extents of the plastic housing of the headset at least 0.1″ away from the heart sensor cable, which helps to reduce the possible electrical interference to an acceptable level.

In some embodiments, the plurality of sensors in the integrated headset can have different types of contacts with the test subject. Here, the contacts can be made of an electrically conductive material, which for non-limiting examples can be but are not limited to, nickel-coated copper or a conductive plastic material. The integrated headset can minimize the noise entering the measuring contact points of the sensors by adopting dry EEG electrodes that work at acceptable noise levels without the use of conductive gels or skin abrasion.

In some embodiments, a non-adhesive or rubber-like substance can be applied against the skin to create a sweat layer between the two that increases the friction between the skin and the headset, normally in less than a minute. This sweating liquid provides better conductivity between the skin and the contacts of the plurality of sensors. In addition, this liquid creates a surface tension that increases the friction and holding strength between the skin and the headset, creating a natural stabilizer for the headset without the use of gels, adhesives or extraneous attachment mechanisms. The holding force increases significantly only in parallel to the plane of the skin, keeping the headset from sliding around on the skin, which is the major problem area in noise generation. Such non-adhesive substance does not, however, significantly increase the holding strength perpendicular to the plane of the skin, so it is not uncomfortable to remove the headset from the tester as it would be the case if an adhesive were applied to hold the headset in place as with many medical sensing devices.

In some embodiments, the headset is operable to promote approximately even pressure distribution at front and back of the person's head to improve comfort and/or produce better signals of the measured physiological data. A foam pad can be used to create a large contact area around the sensors (such as the heart rate sensor 1305) and to create a consistent height for the inside of the headset. This result is increased user comfort since the foam reduces pressure at contact points that would otherwise exist at the raised EEG contacts. It also helps to create the correct amount of pressure at the contact points on the forehead.

Human heads exist in many different shapes and sizes and any headset that is easy to use must accommodate various shapes and sizes of the testers' heads. It is impractical, however, to create numerous different shapes and sizes for the integrated headset as it would require a trained fitter to choose the correct one for each different tester. In addition, the fitting process would be so time-consuming that it defeats the main goal of making the headset easy to use.

In some embodiments, the integrated headset is designed to be adaptive, flexible and compliant, which can automatically adjust to different head shapes and sizes of tester's heads. Since poor contact or movement relative to the skin has the potential to generate a greater amount of noise than the headset can handle, the headset is designed in such a way to minimize movement and to create compliance and fitting to varying head shapes and sizes. The tester should be able to simply put on the headset, tighten the adjustable strap 1308 that allows the headset to be worn comfortably, and be ready to work.

In some embodiments, the compliance in the adjustable strap 1308 of the headset must be tuned so that it is not overly soft and can support weight of the headset; otherwise the headset may result in a situation where the noise from the moving headset would override the measured signal from the sensors. On the other hand, the compliance cannot be so little that it would necessitate over-tightening of the headset, because the human head does not cope well with high amount of pressure being applied directly to the head, which may cause headaches and a sense of claustrophobia on the test subject who wears a headset that is too tight.

In some embodiments, the headset itself surrounds and holds these components on the brow of the head and passes over both ears and around the back of the head. The body of the headset is made of a thin, lightweight material such as plastic or fabric that allows flexing for the headset to match different head shapes but is stiff in the minor plane to not allow twisting, which may cause the electrodes to move and create noise.

In some embodiments, the EEG electrodes and the heart rate sensor both need contacts with the skin of the tester's head that are near the center of the forehead and do not slide around. However, too much contact pressure may create an uncomfortable situation for the tester and is thus not acceptable. Therefore, the integrated headset applies consistent pressure at multiple contact points on different head shapes and sizes of testers, wherein such pressure is both compliant enough to match different head geometries and to create stickiness to the skin and help to stabilize the headset. Here, the headset is operable to achieve such pre-defined pressure by using various thicknesses, materials, and/or geometries at the desired locations of the contact points.

In some embodiments, one or more processing units (1301) that deal with data collection, signal processing, and information transmission are located above the ears to give the unit, the largest component on the headset, a stable base, as allowing the units to hang unsupported would cause them to oscillate with any type of head movement. A silicon stabilization strip 1303 allows for more robust sensing through stabilization of the headset by minimizing movement.

In some embodiments, electronic wiring and/or circuitry (electronic components) of the headset can be placed inside the plastic housing of the headset with another layer of 0.015″ thick ABS plastics in between the electronic components and the skin to provide protection to the components and/or an aesthetic cover for the headset. The inside plastic can be retained by a series of clips and tabs to allow the plastic to slide relative to the outer housing, which precludes the creation of a composite beam if the two were attached together using glue or any other rigid attachment mechanism, as a composite beam is much stiffer than two independent pieces of material and would thus decrease the compliance of the headset.

In some embodiments, the adjustable rubber strip 1308 can be attached to the inside plastic at the very bottom along the entire length of the headset, which creates a large surface area over which an increased friction force may keep the headset from moving. Having consistent and repeatable contact is crucial to the quality of the EEG data and friction increase from the rubber strip facilitates that process. The strip also provides some cushioning which increases user comfort.

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

A method is described for sensing electrical activity in tissue of a user. Electrical activity is detected from the tissue between a first point and a second point on skin of the user and a voltage signal is generated in response thereto which contains a signal of interest and undesired signals. The voltage signal is amplified to amplify the signal of interest and undesired signals without substantially amplifying the noise. The amplification results in an output signal.

Another method is disclosed for sensing electrical activity in tissue of a user in a noise environment that is subjected to electrical noise. A sensor electrode is connected to skin of the user at a first point. A reference electrode is connected to skin of the user at a second point which is in a spaced apart relationship to the first point to allow the sensor electrode to sense the electrical activity in the tissue at the first point relative to the second point. An amplifier is provided which is configured to amplify the electrical activity while substantially reducing the influence from the noise environment.

A sensor circuit is described for sensing electrical activity in tissue of a user and isolating and amplifying a signal of interest from the sensed electrical activity. The sensor circuit includes a sensor electrode for placing on skin of the user at a first point. A reference electrode for placing at a second point which is a distance away from the first point to allow the sensor electrode to sense the electrical activity and to produce a voltage signal relative to the second point which includes the signal of interest in response. An electronic module of the sensor circuit includes a power source with positive and negative source voltages and a source reference voltage which is electrically connected to the reference electrode. An amplifier is connected to receive power from the power source and to receive the voltage signal from the sensor electrode and the power source reference voltage. The amplifier produces an output signal which is proportional to the voltage signal relative to the power source reference voltage. A filter portion receives the output signal from the amplifier and attenuates electrical activity unrelated to the signal of interest while passing the signal of interest.

Embodiments of the systems and methods described herein include a device comprising: at least one sensor and a reference electrode connected to a mounting device; a processor coupled to the sensor and the reference electrode and receiving signals from the sensor, the signals representing electrical activity in tissue of a user, the processor generating an output signal including data of a difference between a first energy level in a first frequency band of the signals and a second energy level in a second frequency band of the signals, wherein the difference is proportional to release level present time emotional state of the user; and a wireless transmitter that transmits the output signal to a remote device.

Embodiments of the systems and methods described herein include an integrated headset, comprising: a power unit operable to provide operating power for the headset; a plurality of sensors operable to measure physiological data from a person wearing the headset, wherein the plurality of sensors include one or more of a motion detection unit operable to sense movement of the head of the person, a heart rate sensor operable to measure heart rate of the person, and a set of electroencephalogram (EEG) electrodes operable to measure EEG signals from the person; a signal processing unit operable to collect, digitize, process, and transmit the physiological data measured from the person to a separate location; an adjustable strap operable to adjust the headset to a comfortable tension setting for the head shape and size of the person; and a stabilizing component operable to stabilize and connect the above components of the headset together.

The powering unit of an embodiment is a rechargeable or replaceable battery.

The physiological data of an embodiment is one or more of: heart rate, brain wave, EEG signal, blink rate, breathing, motion, muscle movement, galvanic skin response, skin temperature, and any other physiological response of the person.

The plurality of sensors of an embodiment further includes one of: an electroencephalogram, a blood oxygen sensor, a galvanometer, an electromygraph, a skin temperature sensor, a breathing sensor, and any other physiological sensor.

One or more of the plurality of sensors of an embodiment are further operable to record the physiological data measured.

The motion detection unit of an embodiment is a three axis accelerometer.

The axes of the accelerometer of an embodiment are aligned closely to regularly accepted axes directions in a three-dimensional space.

The set of EEG electrodes of an embodiment have another contact for a ground reference on one ear of the person.

The set of EEG electrodes of an embodiment are prefrontal dry electrodes that do not need gel or skin preparation to be used.

The set of EEG electrodes of an embodiment are positioned symmetrically about the centerline of forehead of the person.

The heart rate sensor of an embodiment is positioned directly in center of the forehead of the person between the set of EEG electrodes.

The signal processing unit of an embodiment is operable to transmit the measured physiological data wirelessly.

The powering unit and the signal processing unit of an embodiment are positioned over ears of the person, respectively.

The adjustable strap of an embodiment is positioned on rear of the person's head.

The stabilizing component of an embodiment is a silicon stabilization strip.

The stabilizing component of an embodiment is operable to minimize the person's head movement.

The headset of an embodiment is operable to be turned on with a push button and to measure and/or record the physiological data instantly.

The headset of an embodiment is non-intrusive, allowing the person wearing the headset to freely conduct a plurality of functions without any substantial interference from the plurality of sensors integrated in the headset.

The headset of an embodiment is operable to minimize data artifacts arising from at least one or more of: electronic interference among the plurality of sensors, poor contacts between the plurality of sensors and the person's head, and head movement of the person.

The headset of an embodiment is operable to measure the physiological data from the person accurately without requiring any gel or skin preparation at contact points between the plurality of sensors and the person's skin.

Embodiments of the systems and methods described herein include an integrated headset, comprising: means to provide operating power for the headset; means to measure physiological data from a person wearing the headset; means to collect, digitize, process, and transmit the physiological data measured from the person to a separate location; means to adjust the headset to a comfortable tension setting for the head shape and size of the person; and means to stabilize and connect the above components of the headset together.

Embodiments of the systems and methods described herein include an integrated headset, comprising: a power unit operable to provide operating power for the headset; a plurality of sensors operable to measure physiological data from a person wearing the headset; and a signal processing unit operable to collect, digitize, process, and transmit the physiological data measured to a separate location; wherein the headset allows adjustability to fit shape and/or size of the person's head.

The physiological data of an embodiment is one or more of: heart rate, brain waves, electroencephalogram (EEG) signals, blink rate, breathing, motion, muscle movement, galvanic skin response, skin temperature, and any other physiological response of the person.

Each of the plurality of sensors of an embodiment is one of: an electroencephalogram, an accelerometer, an EEG electrode, a heart rate sensor, a blood oxygen sensor, a galvanometer, an electromygraph, a skin temperature sensor, a breathing sensor, and any other physiological sensor.

The system of an embodiment includes a smooth flexible strip operable to promote adhesion to the head by surface tension created by a sweat layer under the strip to stabilize the headset for more robust sensing.

The system of an embodiment includes a foam pad operable to create a large contact area around the plurality of sensors and/or to create a consistent height for the inside of the headset.

The system of an embodiment includes an adjustable strap operable to adjust the headset to a comfortable tension setting for the head shape and size of the person and where the pressure applied to the plurality of sensors is adequate for robust sensing without causing discomfort.

Compliance in the adjustable strap of an embodiment is tuned to be not overly soft and can support weight of the headset.

Compliance in the adjustable strap of an embodiment is large enough not to necessitate over-tightening of the headset.

The adjustable strip of an embodiment is attached to the headset in such way as to create a large surface area over which an increased friction force keeps the headset from moving

The headset of an embodiment surrounds and holds the powering unit, the signal processing unit, and the plurality of sensors on the brow of the head and passes over both ears and around the back of the head of the person.

The body of the headset of an embodiment is made of a thin, lightweight material that allows flexing for the headset to match the head shape and size of the person but is stiff in minor plane to not allow twisting.

The thin, lightweight material of an embodiment is plastic or fabric.

The headset of an embodiment is operable to promote even pressure distribution at front and back of the person's head to improve comfort and/or produce better signals of the measured physiological data.

The headset of an embodiment is operable to apply pre-defined pressure at multiple contact points between the plurality of sensors and the person's skin, wherein such pressure is both compliant enough to match the head geometries of the person and to create stickiness to the skin and help to stabilize the headset.

The headset of an embodiment is operable to achieve the pre-defined pressure via one or more of: various thicknesses, materials, and geometries at desired locations of the contact points.

The headset of an embodiment is operable to minimize data artifacts arising from at least one or more of: electronic interference among the plurality of sensors, poor contacts between the plurality of sensors and the person's head, and movement between the headset and the person's head.

The headset of an embodiment is operable to place signal processing unit over an ear of the person to give the unit a stable base.

The headset of an embodiment is operable to place electronic components of the headset inside a plastic housing of the headset to provide protection to the components and/or an aesthetic cover for the headset without creating a composite beam.

Embodiments of the systems and methods described herein include a method to support measuring physiological data via an integrated headset, comprising: placing the integrated headset on a person, wherein the headset allows adjustability to fit shape and/or size of the person's head; powering operation for the headset via a powering unit in the headset; measuring physiological data from the person wearing the headset via a plurality of sensors in the headset; and collecting, digitizing, processing, and transmitting the physiological data measured to a separate location via a signal processing unit in the headset.

The method of an embodiment includes promoting adhesion to the head by surface tension created by a sweat layer under the strip to stabilize the headset for more robust sensing.

The method of an embodiment includes adjusting the headset to a comfortable tension setting for the head shape and size of the person and where the pressure applied to the plurality of sensors is adequate for robust sensing without causing discomfort.

The method of an embodiment includes promoting even pressure distribution at front and back of the person's head to improve comfort and/or produce better signals of the measured physiological data.

The method of an embodiment includes applying pre-defined pressure at multiple contact points between the plurality of sensors and the person's skin, wherein such pressure is both compliant enough to match the head geometries of the person and to create stickiness to the skin and help to stabilize the headset.

The method of an embodiment includes placing electronic components of the headset inside a plastic housing of the headset to provide protection to the components and/or an aesthetic cover for the headset without creating a composite beam.

Embodiments of the systems and methods described herein include a system to support measuring physiological data via an integrated headset, comprising: means for placing the integrated headset on a person, wherein the headset allows adjustability to fit shape and/or size of the person's head; means for powering operation for the headset via a powering unit in the headset; means for measuring physiological data from the person wearing the headset via a plurality of sensors in the headset; and means for collecting, digitizing, processing, and transmitting the physiological data measured to a separate location via a signal processing unit in the headset.

Embodiments of the systems and methods described herein include an integrated headset, comprising: a power unit operable to provide operating power for the headset; a plurality of sensors operable to measure physiological data from a person wearing the headset; and a signal processing unit operable to collect, digitize, process and transmit the physiological data measured to a separate location; wherein the headset is operable to minimize data artifacts arising from at least one or more of: electronic interference among the plurality of sensors, poor contacts between the plurality of sensors and the person's head, and head movement of the person.

The headset of an embodiment is operable to minimize the electronic interference via one or more of: tuning down power levels of one or more of the plurality of sensors; setting specific distance between signal-carrying wires of the plurality of sensors; separating the signal processing unit and the powering unit physically; and transmitting the measured physiological data wirelessly to minimize 60 Hz noise.

The headset of an embodiment is operable to place signal processing and powering units together or separately over opposite ears of the person, respectively.

The plurality of sensors of an embodiment has different types of contacts with the person.

The contacts of an embodiment are made of an electrically conductive material.

The electrically conductive material of an embodiment is nickel-coated copper or a conductive plastic material.

The plurality of sensors of an embodiment is wired to the signal processing unit in such a way as to minimize the electronic interference.

The system of an embodiment includes an adjustable strap operable to adjust the headset to a comfortable tension setting for the head shape and size of the person;

The system of an embodiment includes a stabilization strip operable to stabilize the headset for more robust sensing.

The physiological data of an embodiment is one or more of: heart rate, brain waves, electroencephalogram (EEG) signals, blink rate, breathing, motion, muscle movement, galvanic skin response, skin temperature, and any other physiological response of the person.

Each of the plurality of sensors of an embodiment is one of: an electroencephalogram, an accelerometer, an EEG electrode, a heart rate sensor, a blood oxygen sensor, a galvanometer, an electromygraph, a skin temperature sensor, a breathing sensor, and any other physiological sensor.

One or more of the plurality of sensors of an embodiment are further operable to record the physiological data measured.

The headset of an embodiment is non-intrusive, allowing the person wearing the headset to freely conduct a plurality of functions without any substantial interference from the plurality of sensors integrated in the headset.

The headset of an embodiment is operable to measure the physiological data from the person accurately without requiring any gel or skin preparation at contact points between the plurality of sensors and the person's skin.

The headset of an embodiment is operable to apply a non-adhesive or rubber-like substance to create a sweat layer between the plurality of sensors and the person's skin to provide better conductivity and/or to increase friction between the skin and the contacts of the plurality of sensors.

Embodiments of the systems and methods described herein include a method to support measuring physiological data via an integrated headset, comprising: placing the integrated headset on a person; powering operation for the headset via a powering unit in the headset; measuring physiological data from the person wearing the headset via a plurality of sensors in the headset to a separate location; and collecting, digitizing, processing, and transmitting the physiological data measured via a signal processing unit in the headset while minimizing data artifacts arising from at least one or more of: electronic interference among the plurality of sensors, poor contacts between the plurality of sensors and the person's head, and head movement of the person.

The method of an embodiment includes minimizing the electronic interference via one or more of: tuning down power levels of one or more of the plurality of sensors; setting specific distance between signal-carrying wires of the plurality of sensors; separating the signal processing unit and the powering unit physically; and transmitting the measured physiological data wirelessly to minimize 60 Hz noise.

The method of an embodiment includes wiring the plurality of sensors to the signal processing unit in such a way as to minimize the electronic interference.

The method of an embodiment includes allowing the person wearing the headset to freely conduct a plurality of functions without any substantial interference from the plurality of sensors integrated in the headset.

The method of an embodiment includes measuring the physiological data from the person accurately without requiring any gel or skin preparation at contact points between the plurality of sensors and the person's skin.

The method of an embodiment includes applying a non-adhesive or rubber-like substance to create a sweat layer between the plurality of sensors and the person's skin to provide better conductivity and/or to increase friction between the skin and the contacts of the plurality of sensors.

Embodiments of the systems and methods described herein include a system to support measuring physiological data via an integrated headset, comprising: means for placing the integrated headset on a person; means for powering operation for the headset via a powering unit in the headset; means for measuring physiological data from the person wearing the headset via a plurality of sensors in the headset; and means for collecting, digitizing, processing, and transmitting the physiological data measured via a signal processing unit in the headset while minimizing data artifacts arising from at least one or more of: electronic interference among the plurality of sensors, poor contacts between the plurality of sensors and the person's head, and head movement of the person.

Embodiments of the systems and methods described herein include an integrated headset, comprising: a power unit operable to provide operating power for the headset; a plurality of sensors operable to measure physiological data from a person wearing the headset; and a processing unit operable to collect, digitize, process and transmit the physiological data measured; wherein the headset is operable to measure the physiological data from the person accurately without requiring any conductive gel or skin preparation at contact points between the plurality of sensors and the person's skin.

The system of an embodiment includes an adjustable strap operable to adjust the headset to a comfortable tension setting for the head shape and size of the person.

The system of an embodiment includes a stabilization strip operable to stabilize the headset for more robust sensing.

The system of an embodiment includes a foam pad operable to create a large contact area around the plurality of sensors and/or to create a consistent height for the inside of the headset.

The headset of an embodiment is operable to apply a non-adhesive or rubber-like substance to create a sweat layer between the plurality of sensors and the person's skin to provide better conductivity and/or to increase friction between the skin and the contacts of the plurality of sensors.

The friction of an embodiment increases significantly only in parallel to plane of the skin, while holding strength perpendicular to the plane of the skin does not significantly increase.

The physiological data of an embodiment is one or more of: heart rate, brain waves, electroencephalogram (EEG) signals, blink rate, breathing, motion, muscle movement, galvanic skin response and any other physiological response of the person.

Each of the plurality of sensors of an embodiment is one of: an electroencephalogram, an accelerometer, an EEG electrode, a heart rate sensor, a blood oxygen sensor, a galvanometer, an electromygraph, a skin temperature sensor, a breathing sensor, and any other physiological sensor.

The headset of an embodiment is operable to adopt dry EEG electrode that works at acceptable noise levels without the use of conductive gel or skin abrasion.

One or more of the plurality of sensors of an embodiment are further operable to record the physiological data measured.

The signal processing unit of an embodiment is operable to transmit the measured physiological data wirelessly.

The headset of an embodiment is non-intrusive, allowing the person wearing the headset to freely conduct a plurality of functions without any interference from the plurality of sensors integrated in the headset.

The headset of an embodiment is operable to minimize data artifacts arising from at least one or more of: electronic interference among the plurality of sensors, poor contacts between the plurality of sensors and the person's head, and movement between the headset and the person's head.

Embodiments of the systems and methods described herein include a method to support measuring physiological data via an integrated headset, comprising: placing the integrated headset on a person; powering operation for the headset via a powering unit in the headset; measuring physiological data accurately from the person wearing the headset via a plurality of sensors in the headset without requiring any conductive gel or skin preparation at contact points between the plurality of sensors and the person's skin; and collecting, digitizing, processing, and transmitting the physiological data measured via a signal processing unit in the headset.

The method of an embodiment comprises creating a large contact area around the plurality of sensors and/or a consistent height for the inside of the headset.

The method of an embodiment comprises applying a non-adhesive or rubber-like substance to create a sweat layer between the plurality of sensors and the person's skin to provide better conductivity and/or to increase friction between the skin and the contacts of the plurality of sensors.

The method of an embodiment comprises adopting dry EEG electrode that works at acceptable noise levels without the use of conductive gel or skin abrasion.

The method of an embodiment comprises adjusting the headset to a comfortable tension setting for the head shape and size of the person.

The method of an embodiment comprises allowing the person wearing the headset to freely conduct his/her normal functions and activities without any interference from the plurality of sensors integrated in the headset.

The method of an embodiment comprises transmitting the measured physiological data wirelessly.

Embodiments of the systems and methods described herein include a system to support measuring physiological data via an integrated headset, comprising: means for placing the integrated headset on a person; means for powering operation for the headset via a powering unit in the headset; means for measuring physiological data accurately from the person wearing the headset via a plurality of sensors in the headset without requiring any conductive gel or skin preparation at contact points between the plurality of sensors and the person's skin; and means for collecting, digitizing, processing, and transmitting the physiological data measured via a signal processing unit in the headset.

Embodiments of the systems and methods described herein include an integrated headset, comprising: a power unit operable to provide operating power for the headset; a plurality of sensors operable to measure physiological data from a person wearing the headset; and a signal processing unit operable to collect, digitize, process, and transmit the physiological data measured to a separate location; wherein the headset is non-intrusive, allowing the person wearing the headset to freely conducting a plurality of functions without any substantial interference from the plurality of sensors integrated in the headset.

The system of an embodiment comprises an adjustable strap operable to adjust the headset to a comfortable tension setting for the head shape and size of the person;

The system of an embodiment comprises a stabilization component operable to stabilize and connect the above components of the headset together.

The powering unit of an embodiment is a rechargeable or replaceable battery.

The physiological data of an embodiment is one or more of: heart rate, brain waves, electroencephalogram (EEG) signals, blink rate, breathing, motion, muscle movement, galvanic skin response and any other physiological response of the person.

Each of the plurality of sensors of an embodiment is one of: an electroencephalogram, an accelerometer, an EEG electrode, a heart rate sensor, a blood oxygen sensor, a galvanometer, an electromygraph, a skin temperature sensor, a breathing sensor, and any other physiological sensor.

Axes of the accelerometer of an embodiment are aligned closely to regularly accepted axes directions in a three-dimensional space.

One or more of the plurality of sensors of an embodiment are further operable to record the physiological data measured.

The signal processing unit of an embodiment is operable to transmit the measured physiological data wirelessly.

The plurality of functions of an embodiment includes watching a plurality of media instances and/or conducting the person's normal activities.

The headset of an embodiment is operable to minimize data artifacts arising from at least one or more of: electronic interference among the plurality of sensors, poor contacts between the plurality of sensors and the person's head, and head movement of the person.

The headset of an embodiment is operable to measure the physiological data from the person accurately without requiring any gel or skin preparation at contact points between the plurality of sensors and the person's skin.

Embodiments of the systems and methods described herein include a method to support measuring physiological data via an integrated headset, comprising: fitting the integrated headset on a person; powering operation for the headset via a powering unit in the headset; measuring physiological data from the person wearing the headset via a plurality of sensors in the headset, while allowing the person to freely conduct a plurality of functions without any substantial interference from the plurality of sensors integrated in the headset; and collecting, digitizing, processing, and transmitting the physiological data measured to a separate location via a signal processing unit in the headset.

The method of an embodiment comprises adjusting the headset to a comfortable tension setting for the head shape and size of the person.

The method of an embodiment comprises transmitting the measured physiological data wirelessly.

The method of an embodiment comprises recording the physiological data measured.

The method of an embodiment comprises collecting, digitizing, processing, and transmitting the physiological data while minimizing data artifacts arising from at least one or more of: electronic interference among the plurality of sensors, poor contacts between the plurality of sensors and the person's head, and head movement of the person.

The method of an embodiment comprises measuring the physiological data accurately from the person without requiring any gel or skin preparation at contact points between the plurality of sensors and the person's skin.

Embodiments of the systems and methods described herein include a system to support measuring physiological data via an integrated headset, comprising: means for fitting the integrated headset on a person; means for powering operation of the headset via a powering unit in the headset; means for measuring physiological data from the person wearing the headset via a plurality of sensors in the headset, while allowing the person to freely conduct a plurality of functions without any substantial interference from the plurality of sensors integrated in the headset; and means for collecting, digitizing, processing, and transmitting the physiological data measured via a signal processing unit in the headset.

The embodiments described herein include and/or run under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, cellular telephones, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components of the systems described herein, and/or provided by some combination of algorithms. The methods described herein can be implemented in one or more of software algorithm(s), programs, firmware, hardware, components, circuitry, in any combination.

The components described herein can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the systems and methods include: microcontrollers with memory (such as electronically erasable programmable read only memory (EEPROM)), embedded microprocessors, firmware, software, etc. Furthermore, aspects of the systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, HTTPs, FTP, SMTP, WAP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the systems and methods is not intended to be exhaustive or to limit the systems and methods to the precise forms disclosed. While specific embodiments of, and examples for, the systems and methods are described herein for illustrative purposes, various equivalent modifications are possible within the scope of the systems and methods, as those skilled in the relevant art will recognize. The teachings of the systems and methods provided herein can be applied to other systems and methods, not only for the systems and methods described above.

The elements and acts of the various embodiments described above can be combined to provide further embodiments. These and other changes can be made to the systems and methods in light of the above detailed description.

What is claimed is:

1. A device comprising:
at least one sensor and a reference electrode connected to a mounting device;
a processor coupled to the sensor and the reference electrode and receiving signals from the sensor, the signals representing electrical activity in tissue of a user, the processor generating an output signal including data of a difference between a first energy level in a first frequency band of the signals and a second energy level in a second frequency band of the signals, wherein the difference is proportional to release level present time emotional state of the user; and
a wireless transmitter that transmits the output signal to a remote device.
